(19) United States
(12) Patent Application Publication
(10) Pub. No.: US 2008/0022467 A1
(43) Pub. Date: Jan. 31, 2008

(54) Disazo dyes and their preparation and use

(75) Inventors: Kurt Baettig, Le Mouret (CH); Gerald Jan, Villars-sur-Glane (CH)

Correspondence Address: Dara L. Onofrio, Esq.;c/o ONOFRIO LAW, 107 Shad Row, Piermont, NY, 10986, US

(73) Assignee: ILFORD Imaging Switzerland GmbH

(21) Appl. No.: 11/881,646

(22) Filed: Jul. 27, 2007

(30) Foreign Application Priority Data
Jul. 27, 2006 (EP) 06118014.7

(51) Int. Cl. C09D 11/02; C07C 245/08; C09B 67/36

(52) U.S. Cl. 8/641; 106/31.48; 534/560; 8/639

(57) ABSTRACT

Disazo dyes of general formula (V)

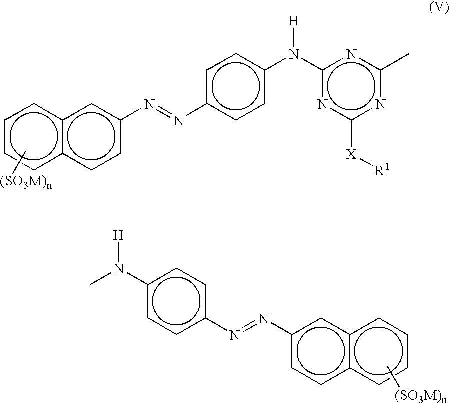

wherein X, R¹, M and n are as defined in the specification, are excellent yellow dyes for dying and printing of cellulose containing materials and textile materials, and, especially, for the preparation of inks for ink jet printing.

### FIELD OF THE INVENTION

The invention relates to novel yellow disazo dyes, their salts, a method of their preparation and their use in dying and printing operations. It relates also to liquid dye preparations containing these dyes, particularly to aqueous recording liquids for ink jet printing.

### BACKGROUND OF THE INVENTION

Ink jet printing processes are essentially of two types:

In continuous stream ink jet printing systems, a recording liquid is emitted in a continuous stream under pressure through a nozzle. The stream breaks up into individual droplets at a certain distance from the nozzle. If a specific location on the recording sheet has to be printed the individual droplets are directed to the recording sheet, otherwise they are directed to a collecting vessel. This is done for example by charging unnecessary droplets in accordance with digital data signals and passing them through an electrostatic field which adjusts the trajectory of these droplets in order to direct them to the collecting vessel. The inverse procedure may also be used wherein uncharged droplets end up in the collecting vessel.

In the non-continuous process, or the so-called “drop-on-demand” systems, droplets are generated in accordance with digital data signals only if a specific location on the recording sheet has to be printed.

The speed of modern ink jet printers has to increase steadily increasing for economic reasons. Digital images, captured with digital cameras or generated by scanning of silver halide camera films, may be printed with these modern printers at a quality level that makes these images nearly indistinguishable from classical copies on silver halide materials. Images produced in this way need to have excellent storage stability even under adverse conditions. This can only be achieved by using a finely tuned system of recording liquids (respectively the dyes contained therein) together with a suitable recording sheet.

Recording sheets suitable for these printers need to absorb the recording liquids very rapidly, in particular during the printing of photo realistic images. Recording sheets particularly suitable for this purpose comprise nanoporous inorganic compounds, preferably oxides, such as aluminum oxides or silicon dioxide, or oxide/hydroxides, such as aluminum oxide/hydroxides. These recording sheets are known as “nanoporous” recording sheets.

Nanoporous recording sheets absorb the recording liquids very rapidly (in the microsecond range) by the action of the capillary forces of the nanoporous compounds. Polymer based recording sheets absorb the recording liquids more slowly (in the millisecond range) by swelling of the polymer.

Most of the commercially available combinations of recording liquids and nanoporous recording sheets do not satisfy all the necessary requirements. The yellow dyes used nowadays do not have all required properties, such as very high brilliance (saturation), a suitable hue, good light stability, good resistance against degradation by ozone, an excellent diffusion fastness. They have to penetrate into the recording sheet and should not show dye aggregation on the surface of the recording sheet (“bronzing”) and they need to have an excellent solubility in the mainly aqueous recording liquid.

Although quite a number of different yellow dyes have already been proposed as dyes for ink jet printing, none meets all the necessary requirements.

The yellow dye of formula (I) is described in patents U.S. Pat. No. 6,277,185 (example 2) and CH 606,321 (example 3).

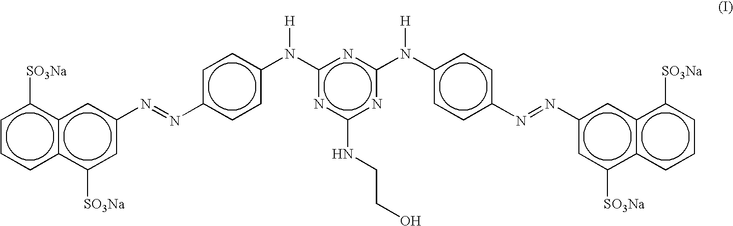

The yellow dye “Direct Yellow 86” of formula (II) is commercially available.

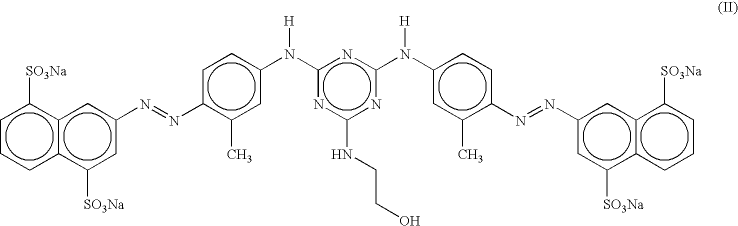

The yellow dye “Direct Yellow 132” of formula (III) is also commercially available.

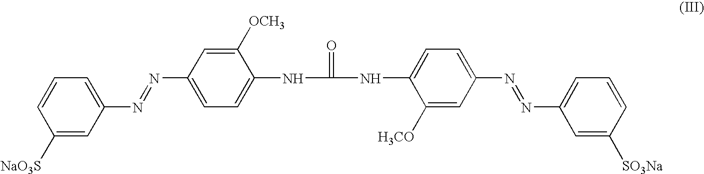

The yellow dye of formula (IV) is described in patent application WO 01/66,651 (example 5).

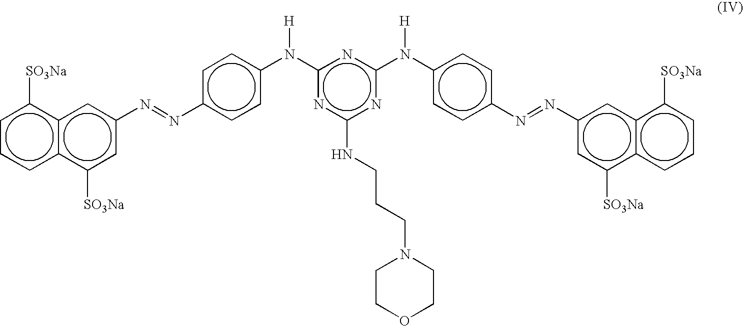

All these dyes mentioned, representing the state of the art, do not satisfy all the required demands if they are used in the formulation of recording liquids for ink jet printing that should provide images or colorings with maximum color rendition and a hue angle of more than 85° on any type of recording sheet as plain or coated paper, coated or uncoated, opaque or transparent synthetic materials.

Dyes used for such recording liquids need to have a high solubility in the essentially aqueous recording liquid, they have to penetrate into the recording sheet and should not show dye aggregation on the surface of the recording sheet (“bronzing”). They need to provide printed images having high optical density, good water fastness, good light stability and good storage stability even in the presence of air pollutants (such as, for example, ozone). They need to be stable in the recording liquid even when the recording liquid is stored for a long time under adverse conditions.

Various types of compositions have been proposed as recording liquids. Typical recording liquids comprise one or more dyes or pigments, water, organic co-solvents and other additives.

The recording fluids have to satisfy the following criteria:
(1) The recording liquid gives images of excellent quality on any type of recording sheet.
(2) The recording liquid gives images exhibiting good water fastness.
(3) The recording liquid gives images exhibiting good light stability.
(4) The recording liquid gives images exhibiting good resistance against degradation by ozone.
(5) The recording liquid gives images exhibiting excellent diffusion fastness.
(6) The recording liquid gives images without dye aggregation on the surface of the recording sheet (“bronzing”).
(7) The recording liquid gives images exhibiting excellent smudge behaviour.
(8) The recording liquid gives images exhibiting excellent storage stability under conditions of high temperature and humidity.
(9) The recording liquid does not clog jetting nozzles of the ink jet printers even when these are kept uncapped while recording is suspended for long periods.
(10) The recording liquid may be stored for long periods without deterioration of its quality.
(11) The physical properties of the recording liquids, such as viscosity, conductivity and surface tension are all within defined ranges well suited for the intended use.
(12) The recording liquid has to be non-toxic, non-flammable and safe.

### SUMMARY OF THE INVENTION

An object of the invention is to provide novel, brilliant disazo dyes with brilliant lemon yellow color, excellent light stability, excellent diffusion fastness, excellent resistance against degradation by ozone and excellent water solubility. They provide images or colorings showing, in particular in ink jet printing onto nanoporous recording sheets, the required yellow hue and excellent resistance against degradation by ozone. They provide images where the sharpness does not or only slightly deteriorate during long periods of storage under conditions of high temperature and humidity. They also have all the other required properties such as high color saturation, excellent light stability and excellent water fastness.

A further object of the invention is the provision of liquid dye preparations, in particular of recording liquids for ink jet printing, showing a spectrally unchanged hue on any type of recording sheet such as plain or coated paper, coated or uncoated, opaque or transparent synthetic materials.

A further object of the invention is the provision of recording liquids satisfying all the requirements mentioned above.

### DETAILED DESCRIPTION OF THE INVENTION

The present invention relates to novel disazo dyes of general formula (V)

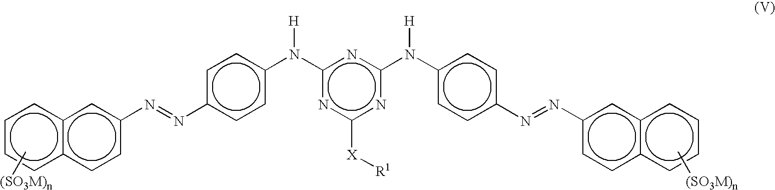

wherein
x represents a sulfur atom or an imino group optionally substituted by an alky group having from 1 to 8 carbon atoms;
R¹ represents a sulfoalkyl group —(CH₂)ₘSO₃M or a 3,5-dicarboxyphenyl group;
M represents a hydrogen atom, a metal cation or an ammonium cation, which may be substituted by one or more alkyl or substituted alkyl or hydroxyalkoxyalkyl groups each having from 1 to 8 carbon atoms;
m is 2, 3 or 4
   and
n is 2 or 3.

Preferred are dyes of general formula (V), wherein
X, M and m are as defined above,
R¹ represents a sulfoalkyl group —(CH₂)ₘSO₃M and
n is equal to 2.

Particularly preferred are dyes of general formula (V), wherein
X, R¹, M and m are as defined above and
the SO₃M substituents are in positions 4,8 or 6,8.

As substituent M, the cations of the alkali metals lithium, sodium and potassium are preferred, as well as the ammonium cation substituted by a hydroxy containing alkyl group having from 1 to 5 carbon atoms. Dyes having incorporated such a substituted ammonium cation show a particularly elevated The prepared dyes of general formula (V) are listed in Table 1 together with the position of their absorption maximum in aqueous solution.

TABLE 1

| Dye | Positions | | | | λₘₐₓ in H₂O |
|---|---|---|---|---|---|
| Nor. | of SO₃M | X | R¹ | M | (nm) at pH 7.0 |
| 10 | 4,8 | S | —(CH₂)₃SO₃M | Na | 370 |
| 11 | 4,8 | NH | —(CH₂)₂SO₃M | Na | 396 |
| 12 | 4,8 | NH | —(CH₂)₂SO₃M | Li | 393 |
| 13 | 4,8 | NH | —(CH₂)₂SO₃M | K | 392 |
| 14 | 4,8 | NCH₃ | —(CH₂)₂SO₃M | Na | 399 |
| 15 | 6,8 | S | —(CH₂)₃SO₃M | Na | 363 |
| 16 | 6,8 | NH | —(CH₂)₂SO₃M | Na | 393 |
| 17 | 6,8 | NCH₃ | —(CH₂)₂SO₃M | Na | 395 |
| 18 | 4,8 | NH | 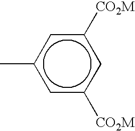 | Na | 389 |

The invention also relates to a method of preparation of the dyes of general formula (V) according to the invention, characterized by the fact that two units of the chromophore of general formula (VI),

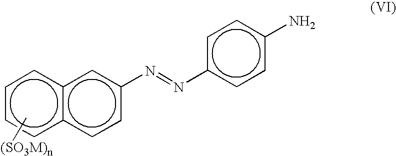

wherein M and n are as defined above,

are condensed with one unit of cyanuric chloride in the presence of a base under formation of the intermediate dye compound of general formula (VII)

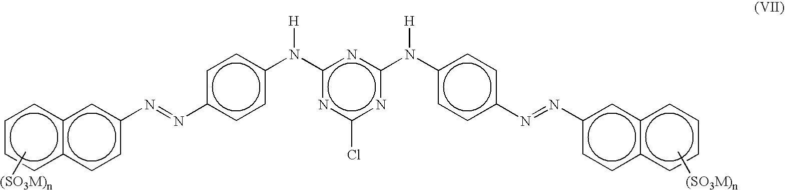

and

this intermediate dye compound of general formula (VII) is reacted subsequently with a compound of formula (VIII)

HX—(CH₂)ₘSO₃M (VIII)

wherein X, M and m are as defined above,

or of general formula (IX),

1,3,5-H₂NC₆H₃(CO₂M)₂ (IX)

wherein M is as defined above,

under conditions that the dyes of general formula (V) are formed.

The disazo dyes of general formula (V) may be in the free acid form or in the form of inorganic or organic salts thereof. Preferably, they are in the form of their alkali or ammonium salts, wherein the ammonium cation may be substituted. Examples of such substituted ammonium cations are 2-hydroxyethylammonium, bis-(2-hydroxyethyl)-ammonium, tris-(2-hydroxyethyl)-ammonium, bis-(2-hydroxyethyl)-methylmmonium, tris-[2-(2-methoxyethoxy)-ethyl]-ammonium, 8-hydroxy-3,6-dioxactylammonium, 1,8-diazabicyclo(5.4.0)undec-7-en, 1,4-diazabicyclo-(2.2.2)-octane and tetraalkylammonium such as tetramethylammonium or tetrabutylammonium, or pyridinium.

The invention does not only relate to pure disazo dyes of general formula (V), but also to mixtures of these disazo dyes.

The disazo dyes of general formula (V) according to the invention are used for dying cellulose containing materials, paper, cotton, viscose, leather and wool to provide dyed materials with good water fastness and light stability.

All methods well known in the textile and paper industries for dyeing with substantive dyes may be used with the disazo dyes, preferably for the bulk or surface treatment of sized or unsized paper. The dyes may also be used in the dyeing of yarns and piece goods of cotton, viscose and linen by the exhaustion process from a long liquor or in a continuous process.

The invention furthermore relates to liquid dye preparations comprising at least one disazo dye of general formula (V) according to the invention. The use of such liquid dye preparations is particularly preferred for paper dyeing. Such stable, liquid, preferably aqueous, concentrated dye preparations may be obtained by using methods well known in the art, preferably by dissolving in suitable solvents. The possibility of preparation of such stable, aqueous, concentrated preparations in the course of dye synthesis itself, without intermediate isolation of the dye, for example after a desalting step by diafiltration of the reaction solution, is of particular advantage.

Such liquid dye preparations may not only contain disazo dyes of general formula (V), but, in addition, other yellow dyes such as Acid Yellow 17, Acid Yellow 23, Direct Yellow 132, Direct Yellow 86 or yellow dyes as described in patent applications WO 96/24,635 and EP 0,755,984.

Patent application WO 96/24,635 describes yellow dyes of general formula (X),

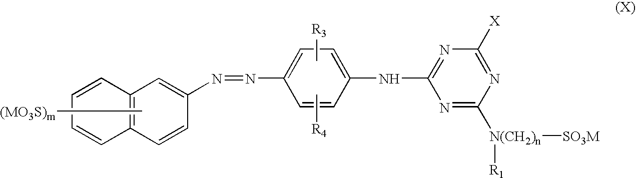

wherein
M represents a hydrogen atom, a monovalent metal cation, an ammonium cation, which may be substituted by one or more alkyl or hydroxy substituted alkyl or hydroxyalkoxyalkyl groups each having from 1 to 12 carbon atoms;
R₁ represents a hydrogen atom or an aliphatic alky group having from 1 to 6 carbon atoms;
R₃, R₄ independently represent a hydrogen atom, an aliphatic alky group, an alkoxyl group or an acylamino group, each having up to 3 carbon atoms;
X represents NR₅R₆,
   wherein
   R₅, R₆ independently represent a hydrogen atom, an alky group having from 1 to 6 carbon atoms, a substituted alkylr group having from 2 to 6 carbon atoms, wherein the substituents are selected from the group consisting of OH, OCH₃, COOM and SO₃M; an aralkyl group, an aryl group or an aryl group substituted by COOM or SO₃M; or where R₅ and R₆ form a ring with or without inclusion of a hetero atom;
or
x represents SR₇,
   wherein
   R₇ represents an unsubstituted alkyl group having from 1 to 6 carbon atoms, a substituted alkyl group having from 2 to 6 carbon atoms, wherein the substituents are selected from the group consisting of OH, OCH₃, COOM and SO₃M;
or
X represents OR₈,
   wherein
   R₈ represents a hydrogen atom or an aliphatitic alkyl group having from 1 to 6 carbon atoms
and
m is 1, 2 or 3 and the substituents SO₃M are in positions 4,8; 5,7; 6, 8; 3, 6, 8; or 4,6,8, and, in the case where m is equal to 1, the substituent SO₃M may be in positions 1 or 4 to 8.

Patent application EP 0,755,984 describes yellow dyes of general formula (XI),

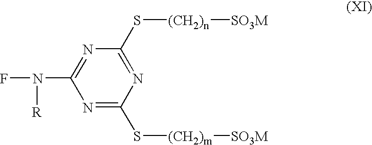

wherein
F represents a monoazo dye residue;
R represents a hydrogen atom or an aliphatitic alkyl group having from 1 to 6 carbon atoms;
M represents a hydrogen atom, a monovalent metal cation, an ammonium cation, which may be substituted by one or more alkyl or hydroxy substituted alkyl or hydroxyalkoxyalkyl groups each having from 1 to 12 carbon atoms
and
m, n independently are from 2 to 6.

The disazo dyes or mixtures of disazo dyes of general formula (V) are excellent dyes for the preparation of yellow recording liquids for ink jet printing. These recording liquids may also contain other yellow dyes such as Acid Yellow 17, Acid Yellow 23, Direct Yellow 132, Direct Yellow 86 or yellow dyes as described in patent applications WO 96/24,635 and EP 0,755,984.

Such a yellow recording liquid comprises one or more of the disazo dyes according to the invention in a liquid aqueous medium and, optionally, other yellow dyes. The recording liquid contains from 0.5% to 20% by weight, preferably from 0.5% to 8% by weight, of these diazo dyes according to the invention, based on the total weight of the recording liquid. The liquid medium is preferably water or a mixture of water and water-miscible organic solvents. Suitable solvents are given for example in patents U.S. Pat. No. 4,626,284, U.S. Pat. No. 4,703,113 and U.S. Pat. No. 4,963,189 and in patent applications GB 2,289,473, EP 0,425,150 and EP 0,597,672.

Particularly preferred is a method of printing of textile materials with such a yellow recording liquid, wherein, in a first step, the ink is deposited on the textile material with the aid of an ink jet printer and, in a second step, the dye is fixed to the textile material by heating to a temperature from 60° C. to 130° C.

The present invention will be illustrated in more detail by the following examples without limiting the scope of the claimed disazo dyes in any way.

### EXAMPLES

### Example 1

### Preparation of the Intermediate Dye Compound

The intermediate dye compound of formula (XII)

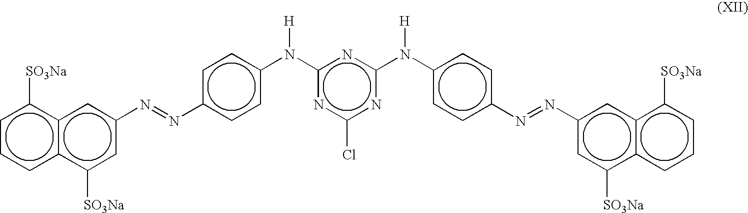

was prepared in the following way:

614 g of the disodium salt of 3-[(4-aminophenyl)azo]-naphthalene-1,5-disulfonic acid (content 77.5%, 1.054 moles) were dissolved in 4900 ml of water at a temperature of 65° C. The warm solution (65° C.) was filtered through 20 g of silica gel under pressure. pH was adjusted to a value between 7.0 and 8.0. Then, the filtering device was rinsed with 100 ml of warm water and the filtrate was cooled down to a temperature of 0° C. 92.8 g of cyanuric chloride (0.504 moles) (available from Fluka Chemie GmbH, Buchs, Switzerland) were added portion-wise at a temperature between 0° C. and 50° C. while maintaining the value of pH between 4.0 and 6.0 by simultaneous addition of an aqueous solution (30%) of sodium hydroxide. The reaction mixture was warmed up to room temperature and finally heated to a temperature of 60° C. while maintaining the value of pH between 6.5 and 7.0 by simultaneous addition of an aqueous solution (30%) of sodium hydroxide. Stirring was continued for 2 hours at a temperature of 60° C. Afterwards, pH was adjusted to a value between 7.0 and 7.2 and the mixture was cooled down to a temperature of 20° C., and, finally, to a temperature of 5° C. The precipitate was vacuum filtered and vacuum dried at a temperature of 50° C. 573 g of the intermediate dye compound of formula (XII) were obtained in this way.

### Preparation of the Disazo Dye No. 10

57 g (0.05 moles) of the intermediate dye compound of formula (XII) were suspended under stirring in 360 ml of water. Afterwards, 14.4 g of the sodium salt of 3-mercapto-propane-1-sulfonic acid, (available from Raschig GmbH, Ludwigshafen, Germany) (content 93%) were added and pH was adjusted to a value of 10.0 by addition of an aqueous solution (30%) of sodium hydroxide. Afterwards, 5.3 g of sodium carbonate were added to the mixture, the mixture was heated to a temperature of 80° C. and stirred for 5 hours at this temperature. pH was adjusted to a value of 6.5 by addition of acetic acid and the solution was clear filtered. The solution was cooled down to a temperature of 20° C. and the dye was precipitated by the addition of sodium acetate and methanol. The mixture was stirred for 2 hours and the precipitate was isolated by vacuum filtration. The precipitate was washed with a mixture (1:1 by volume) of water and methanol and finally vacuum dried at a temperature of 60° C. 35 g of the disazo dye No. 10 were obtained in this way.

### Example 2

### Preparation of the Disazo Dye No. 11

114 g (0.10 moles) of the intermediate dye compound of formula (XII) of example 1 were suspended under stirring in 300 ml of water. The suspension was stirred for 30 minutes at a temperature of 20° C. Afterwards, a mixture of 17.5 g of taurine (0.14 moles), 280 ml of water and 16 g of an aqueous solution (30%) of sodium hydroxide and 16.0 g of sodium carbonate was added and the mixture was heated under reflux to a temperature of 100° C. The turbidity of the mixture disappears at a temperature of 70° C. The mixture was stirred for 2 hours at a temperature of 100° C. and afterwards cooled down to a temperature of 70° C. pH was adjusted to a value between 5.5 and 6.0 by addition of acetic acid. Stirring of the mixture was continued overnight. The orange precipitate was isolated by vacuum filtration.

The wet precipitate was suspended in 600 ml of water and the mixture was heated to a temperature of 70° C. pH was adjusted to a value of 8.0 by addition of an aqueous solution (30%) of sodium hydroxide. Afterwards, 4 g of silica gel were added to the turbid solution. The mixture was clear filtered at a temperature of 600° C. The filtering device was rinsed with 20 ml of water. 860 ml of ethanol (95%) were added drop-wise to the filtrate at a temperature of 60° C. The mixture was cooled down to a temperature of 5° C. and stirred for 1 hour at this temperature. The precipitate was vacuum filtered. The precipitate was washed with 140 ml of ethanol (95%) and finally vacuum dried at a temperature of 70° C. 112 g of the disazo dye No. 11 were obtained in this way.

The disazo dyes No. 12 to No. 18 according to the invention may be prepared in a similar way by using appropriate starting materials.

### Example of Recording Liquid Preparation

The present invention, as far as it relates to recording liquids, is illustrated by the following examples using the disazo dyes of Table 1 according to the invention and dyes representing the state of the art. For each dye, 100 g of recording liquid were prepared by heating the necessary amount of dye (2.0 g-5.0 g), 6.0 g of ethylene glycol, 3.0 g of propylene-1,2-glycol, 3.0 g of 1-methyl-2-pyrrolidone, 0.3 g of an aqueous solution (50%) of Olin® 10G (available from Arch Chemicals Inc., Norwalk, USA), 0.3 g of Surfinol® 465 (available from Air Products and Chemicals Inc., Allentown, USA) and 0.1 g of Mergal® K 10N (available from Riedel-de-Haën, Seelze, Germany) together with water at a temperature of 50° C. under stirring for approximately 1 hour. The resulting solution was cooled down to a temperature of 20° C., its value of pH was adjusted to 7.5 and the solution was passed through a Millipore® filter of 0.3 μm pore diameter. The dye quantity was adjusted in such a way that the optical density of the printed image was similar for all dyes.

### Image Preparation

Homogeneously colored square patches with an approximate density of 1.40 were then printed, using these recording liquids, with an ink jet printer Canon BJC 8500 onto the following recording sheets:
A: HP Printing Paper HPP 1122 (paper)
B: HP Premium Plus (polymer based recording sheet)
C: ILFORD Galerie Smooth Glossy (nanoporous recording sheet)
D: ILFORD Printasia Premium Photo Glossy Paper (nanoporous recording sheet)
E: ILFORD Galerie Classic Gloss Paper (polymer based recording sheet)
F: EPSON Premium Glossy Photo Paper (nanoporous recording sheet)
G: CANON Photo Paper Pro PR-101 (nanoporous recording sheet)

These colored patches were used for the determination of light stability, dye saturation and resistance against degradation by ozone.

### Tests

### 1. Dye Solubility

Dye solubility was determined with a spectrophotometer in the UV and in the visible spectral region. The spectra of the pure disazo dyes according to the invention were measured in a buffered aqueous solution at a value of pH of 7.0. Saturated aqueous solutions of the dyes were prepared at a temperature of 50° C. These solutions were afterwards cooled down to room temperature and diluted for the measurements after a waiting time of 24 hours in such a way that the absorption at the absorption maximum was between 0.8 and 1.5. This dilution factor allows the calculation of dye solubility.

### 2. Color Saturation

The color coordinates L*a*b* of the colored square patches were measured with a spectrophotometer Spectrolino® (available from Gretag Macbeth, Regensdorf, Switzerland) in reflection (illuminant D₆₅). The saturation C* is determined from measured color coordinates according to the expression

C*=\sqrt{a*²+b*²}

### Brilliant dyes have a high value of C*.

### 3. Light Stability

The printed samples were irradiated using a Weather-Ometer® Ci35A (available from Atlas Material Testing Technology, Chicago, USA) with a 6500 W xenon lamp at a temperature of 20° C. and relative humidity of 50% until an illumination of 20 megalux hours was reached. The density loss was measured with a densitometer Spectrolino®. The percent density loss of initial density gives an indication of the light stability of the dyes on the printed recording sheet.

### 4. Dye Diffusion

For this test, colored square patches of 1 cm² of integral density 1.4 containing a finely graded grid were printed with a Canon BJC 8500 ink jet printer, wherein the squares of this grid had a length of 8 pixels and the lines a width of 2 pixels. The printed samples were dried for 24 hours at a relative humidity of the air of 59%. The optical density of the colored square patches was measured with a Spectrolino® densitometer. The printed samples were then stored for 7 days in a climatic test cabinet WK 111 (available from Weiss Umwelttechnik GmbH, Reiskirchen-Lindenstruth, Germany) at a temperature of 40° C. and relative humidity of 80%. During storage, dye may diffuse into the white parts of the grid, giving a density increase. The samples were re-measured after storage. The density increase in percent before and after storage is a measure of the amount of dye diffusion.

### 5. Stability Against Degradation by Ozone

The optical density of the colored square patches was measured with a Spectrolino® densitometer. Afterwards, the printed samples were stored for a predetermined time (for example 48 hours) in an ozone chamber, model 903 (available from Satra/Hampden, Great Britain) at a temperature of 30° C., a relative humidity of the air of 50% and an ozone concentration of 1 ppm at a velocity of the circulating, ozone containing air of 13 mm/s. After storage, the samples were re-measured. The density differences of these two measurements, expressed as per cent of the initial density, are an indication of the amount of dye loss due to the exposure to ozone.

### Results

For each of the above tests, the dyes may be classified into 4 classes:
A: excellent
B: good
C: satisfactory
D: unsatisfactory

The determined solubility of the disazo dyes according to the invention and a few dyes representing the state of the art is listed in Table 2.

TABLE 2

| Dye No. | Weight percent | Class |
|---|---|---|
| 10 | 26 | A |
| 11 | 17 | B |
| 14 | 18 | B |
| 15 | 29 | A |
| 16 | 26 | A |
| 17 | 27 | A |
| 18 | 24 | A |
| I | 16 | C |
| II | 16 | C |
| III | 12 | D |
| IV | 26 | A |

A comparison of the results in Table 2 immediately shows that all the disazo dyes according to the invention have an excellent or a good solubility. Only dye (IV), representing the state of the art, has en equally high water solubility.

The determined color saturation C* of the disazo dyes according to the invention and a few dyes representing the state of the art are listed in Table 3.

TABLE 3

| | Color Saturation C* (Class) | | | | | | |
|---|---|---|---|---|---|---|---|
| | on Recording Sheet | | | | | | |
| Dye No. | A | B | C | D | E | F | G |
| 10 | A | A | A | A | A | A | A |
| 11 | A | A | A | A | A | A | A |
| 14 | A | A | A | A | A | A | A |
| 15 | B | B | B | B | C | B | B |
| 16 | C | B | A | B | C | A | B |
| 17 | D | B | A | C | C | B | B |
| 18 | C | A | A | A | A | A | A |
| I | B | A | A | A | A | A | A |
| II | D | D | D | D | D | D | D |
| III | A | A | A | A | A | A | A |
| IV | C | B | C | B | B | A | B |

A comparison of the results in Table 3 immediately shows that, in particular, the disazo dyes No. 10, 11 and 14 according to the invention are very brilliant and therefore give brilliant yellow colors on all recording sheets. None of the dyes representing the state of the art gives such brilliant yellow colors on all recording sheets.

The measured light stabilities of the disazo dyes according to the invention and a few dyes representing the state of the art are listed in Table 4.

TABLE 4

| | Light Stability (Class) | | | | | | |
|---|---|---|---|---|---|---|---|
| | on Recording Sheet | | | | | | |
| Dye No. | A | B | C | D | E | F | G |
| 10 | B | A | A | B | B | B | B |
| 11 | B | A | A | B | B | A | A |
| 14 | B | A | A | B | B | B | B |
| 15 | B | A | B | B | B | B | B |
| 16 | B | A | B | B | B | B | B |
| 17 | B | A | A | B | B | B | B |
| 18 | B | A | B | B | B | B | B |
| I | A | A | B | B | B | A | B |
| II | B | D | B | B | B | A | A |
| III | C | A | B | B | B | B | B |
| IV | C | A | D | C | C | B | B |

A comparison of the results in Table 4 immediately shows that all disazo dyes according to the invention have an excellent or good light stability on all recording sheets. Only dye (I), representing the state of the art, has an excellent or good light stability on all recording sheets. The dyes (II), (III) and (IV), representing the state of the art, have a satisfactory or even unsatisfactory light stability on all recording sheets. They cannot therefore be used without restrictions in recording liquids for ink jet printing.

The measured amounts of dye diffusion of the disazo dyes according to the invention and a few dyes representing the state of the art are listed in Table 5.

TABLE 5

| | Dye Diffusion (Class) | | | | | | |
|---|---|---|---|---|---|---|---|
| | on Recording Sheet | | | | | | |
| Dye No. | A | B | C | D | E | F | G |
| 10 | A | C | B | B | A | A | A |
| 11 | A | B | B | A | A | A | A |
| 14 | A | B | B | A | B | A | A |
| 15 | A | C | C | B | B | B | B |
| 16 | A | C | A | A | B | A | A |
| 17 | A | C | B | A | B | B | B |
| 18 | A | C | B | A | A | A | A |
| I | B | C | C | A | C | C | A |
| II | A | D | C | B | C | C | A |
| III | A | D | C | C | D | D | A |
| iV | A | C | B | A | B | A | B |

A comparison of the results in Table 5 immediately shows that, in particular, the disazo dyes No. 11 and No. 14 according to the invention have an excellent or good diffusion fastness on all recording sheets. They may therefore be used without restrictions in recording liquids for ink jet printing. All dyes representing the state of the art have a satisfactory or even unsatisfactory diffusion fastness on at least one recording sheet. They cannot therefore be used without restrictions in recording liquids for ink jet printing.

The resistance against degradation by ozone of the disazo dyes according to the invention and a few dyes representing the state of the art is listed in Table 6. Because degradation by ozone is a problem only for nanoporous recording sheets, results are listed only for recording sheets C, D, F and G.

TABLE 6

Resistance against Degradation by Ozone
(Class) on Recording Sheet

| Dye No. | C | D | F | G |
|---|---|---|---|---|
| 10 | A | A | A | A |
| 11 | A | A | A | A |
| 14 | A | A | A | A |
| 15 | B | A | A | A |
| 16 | A | A | A | A |
| 17 | B | A | A | A |
| 18 | A | A | A | A |
| I | A | B | A | A |
| II | A | B | A | A |
| III | D | B | C | C |
| IV | A | B | A | A |

A comparison of the results in Table 6 immediately shows that all disazo dyes according to the invention have an excellent or good resistance against degradation by ozone on all recording sheets. They may therefore be used without restrictions in recording liquids for ink jet printing.

Finally, variations from the examples given herein are possible in view of the above disclosure. Therefore, although the invention has been described with reference to certain preferred embodiments, it will be appreciated that other dyes may be devised, which are nevertheless within the scope and spirit of the invention as defined in the claims appended hereto.

The foregoing description of various and preferred embodiments of the present invention has been provided for purposes of illustration only, and it is understood that numerous modifications, variations and alterations may be made without departing from the scope and spirit of the invention as set forth in the following claims.

1. Disazo dyes of general formula (V) 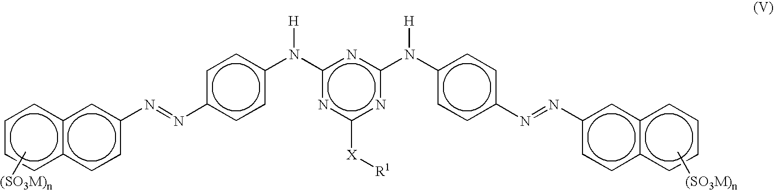
wherein
X represents a sulfur atom or an imino group optionally substituted by an alky group having from 1 to 8 carbon atoms;
R¹ represents a sulfoalkyl group —(CH₂)ₘSO₃M or a 3,5-dicarboxyphenyl group;
M represents a hydrogen atom, a metal cation or an ammonium cation, which may be substituted by one or more alkyl or substituted alkyl or hydroxyalkoxyalkyl groups each having from 1 to 8 carbon atoms;
m is 2, 3 or 4
and
n is 2 or 3.

2. Disazo dyes according to claim 1, characterized by the fact that
R¹ represents a sulfoalkyl group —(CH₂)ₘSO₃M
and
n is equal to 2.

3. Disazo dyes according to claim 2, characterized by the fact that the substituents SO₃M are in positions 4,8 or 6,8.

4. Disazo dyes according to one of claims 2 or 3, characterized by the fact that
M represents a cation of the alkali metals lithium, sodium and potassium or an ammonium cation substituted by an alkyl group or a hydroxy containing alkyl group, each having from 1 to 5 carbon atoms.

5. Method of preparation of the disazo dyes according to claim 1, characterized by the fact that
two units of the chromophore of general formula (VI), 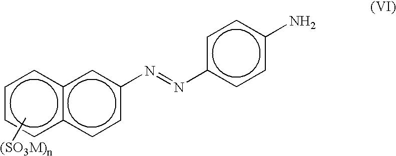
wherein M and n are as defined in claim 1,
are condensed with one unit of cyanuric chloride in the presence of a base under formation of the intermediate dye compound of general formula (VII) 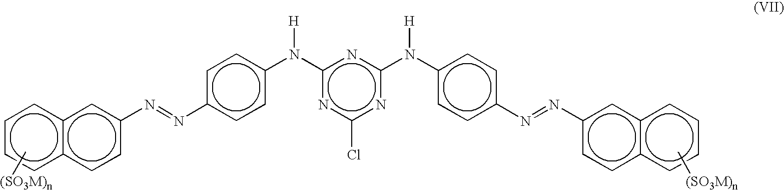
and
this intermediate dye compound of general formula (VII) is reacted subsequently with a compound of formula (VIII)
HX—(CH₂)ₘSO₃M (VIII)
wherein X, M and m are as defined in claim 1,
or of general formula (IX),
1,3,5-H₂NC₆H₃(CO₂M)₂ (IX)
wherein M is as defined in claim 1,
under conditions that the dyes of general formula (V) are formed.

6. Process for recording text and images on recording sheets and for dying and printing natural or synthetic fiber materials, nanoporous materials, leather and aluminum by applying thereto a disazo dye according to one or more of claims 1 to 4.

7. Liquid dye preparations comprising at least one disazo dye or a mixture of disazo dyes according to one or more of claims 1 to 4.

8. Liquid dye preparations according to claim 7, comprising in addition one or more other yellow dyes.

9. Liquid dye preparations according to claim 8, characterized by the fact that the additional yellow dyes are selected from the group consisting of dyes of general formula (X), 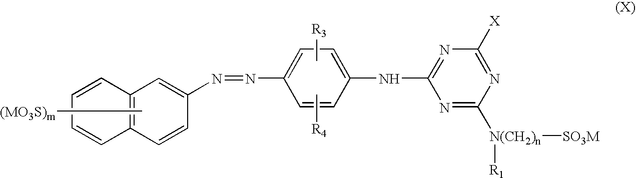
wherein
M represents a hydrogen atom, a monovalent metal cation, an ammonium cation, which may be substituted by one or more alkyl or hydroxy substituted alkyl or hydroxyalkoxyalkyl groups each having from 1 to 12 carbon atoms;
R₁ represents a hydrogen atom or an aliphatic alky group having from 1 to 6 carbon atoms;
R₃, R₄ independently represent a hydrogen atom, an aliphatic alky group, an alkoxyl group or an acylamino group, each having up to 3 carbon atoms;
X represents NR₅R₆,
wherein
R₅, R₆ independently represent a hydrogen atom, an alky group having from 1 to 6 carbon atoms, a substituted alkyl group having from 2 to 6 carbon atoms, wherein the substituents are selected from the group consisting of OH, OCH₃, COOM and SO₃M; an aralkyl group, an aryl group or an aryl group substituted by COOM or SO₃M; or where R₅ and R₆ form a ring with or without inclusion of a hetero atom;
or
x represents SR₇,
wherein
R₇ represents an unsubstituted alkyl group having from 1 to 6 carbon atoms, a substituted alkyl group having from 2 to 6 carbon atoms, wherein the substituents are selected from the group consisting of OH, OCH₃, COOM and SO₃M;
or
x represents OR₈,
wherein
R₈ represents a hydrogen atom or an aliphatic alkyl group having from 1 to 6 carbon atoms
and
m is 1, 2 or 3 and the substituents SO₃M are in positions 4,8; 5,7; 6, 8; 3, 6, 8; or 4,6,8, and, in the case where m is equal to 1, the substituent SO₃M may be in positions 1 or 4 to 8,
of general formula (XI), 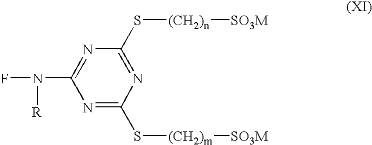
wherein
F represents a monoazo dye residue;
R represents a hydrogen atom or an aliphatic alkyl group having from 1 to 6 carbon atoms;
M represents a hydrogen atom, a monovalent metal cation, an ammonium cation, which may be substituted by one or more alkyl or hydroxy substituted alkyl or hydroxyalkoxyalkyl groups each having from 1 to 12 carbon atoms
and
m, n independently are from 2 to 6,
Acid Yellow 17, Acid Yellow 23, Direct Yellow 132, Direct Yellow 86 or their mixtures.

10. Recording fluid for ink jet printing, comprising at least one disazo dye or a mixture of disazo dyes according to one or more of claims 1 to 4.

11. Recording fluid for ink jet printing according to claim 10, comprising in addition one or more other yellow dyes.

12. Recording fluid for ink jet printing according to claim 11, characterized by the fact that the additional yellow dyes are selected from the group consisting of dyes of general formula (X), 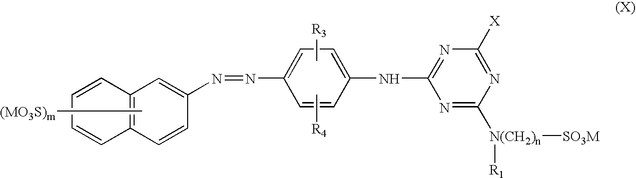
wherein
M represents a hydrogen atom, a monovalent metal cation, an ammonium cation, which may be substituted by one or more alkyl or hydroxy substituted alkyl or hydroxyalkoxyalkyl groups each having from 1 to 12 carbon atoms;
R₁ represents a hydrogen atom or an aliphatic alky group having from 1 to 6 carbon atoms;
R₃, R₄ independently represent a hydrogen atom, an aliphatic alky group, an alkoxyl group or an acylamino group, each having up to 3 carbon atoms;
X represents NR₅R₆,
wherein
R₅, R₆ independently represent a hydrogen atom, an alky group having from 1 to 6 carbon atoms, a substituted alkyl group having from 2 to 6 carbon atoms, wherein the substituents are selected from the group consisting of OH, OCH₃, COOM and SO₃M; an aralkyl group, an aryl group or an aryl group substituted by COOM or SO₃M; or where R₅ and R₆ form a ring with or without inclusion of a hetero atom;
or
X represents SR₇,
wherein
R₇ represents an unsubstituted alkyl group having from 1 to 6 carbon atoms, a substituted alkyl group having from 2 to 6 carbon atoms, wherein the substituents are selected from the group consisting of OH, OCH₃, COOM and SO₃M;
or
X represents OR₈,
wherein
R₈ represents a hydrogen atom or an aliphatic alkyl group having from 1 to 6 carbon atoms
and
m is 1, 2 or 3 and the substituents SO₃M are in positions 4,8; 5,7; 6, 8; 3, 6, 8; or 4,6,8, and, in the case where m is equal to 1, the substituent SO₃M may be in positions 1 or 4 to 8,
of general formula (XI), 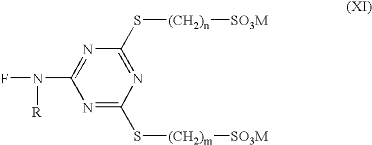
wherein
F represents a monoazo dye residue;
R represents a hydrogen atom or an aliphatic alkyl group having from 1 to 6 carbon atoms;
M represents a hydrogen atom, a monovalent metal cation, an ammonium cation, which may be substituted by one or more alkyl or hydroxy substituted alkyl or hydroxyalkoxyalkyl groups each having from 1 to 12 carbon atoms
and
m, n independently are from 2 to 6,
Acid Yellow 17, Acid Yellow 23, Direct Yellow 132, Direct Yellow 86 or their mixtures.
